SEQUENCE LISTING (3 sequences)

SEQ ID NO: 1 - protein, 690 aa, Homo sapiens
MKRLPLLVVFSTLLNCSYTQNCTKTPCLPNAKCEIRNGIEACYCNMGFSGNGVTICEDDNECGNLTQSCGENANCTNTEGSYYCMCVPGFRSSSNQDRFITNDGTVCIENVNANCHLDNVCIAANINKTLTKIRSIKEPVALLQEVYRNSVTDLSPTDIITYIEILAESSSLLGYKNNTISAKDTLSNSTLTEFVKTVNNFVQRDTFVVWDKLSVNHRRTHLTKLMHTVEQATLRISQSFQKTTEFDTNSTDIALKVFFFDSYNMKHIHPHMNMDGDYINIFPKRKAAYDSNGNVAVAFVYYKSIGPLLSSSDNFLLKPQNYDNSEEEERVISSVISVSMSSNPPTLYELEKITFTLSHRKVTDRYRSLCAFWNYSPDTMNGSWSSEGCELTYSNETHTSCRCNHLTHFAILMSSGPSIGIKDYNILTRITQLGIIISLICLAICIFTFWFFSEIQSTRTTIHKNLCCSLFLAELVFLVGINTNTNKLFCSIIAGLLHYFFLAAFAWMCIEGIHLYLIVVGVIYNKGFLHKNFYIFGYLSPAVVVGFSAALGYRYYGTTKVCWLSTENNFIWSFIGPACLIILVNLLAFGVIIYKVFRHTAGLKPEVSCFENIRSCARGALALLFLLGTTWIFGVLHVVHASVVTAYLFTVSNAFQGMFIFLFLCVLSRKIQEEYYRLFKNVPCCFGCLR

SEQ ID NO: 2 - protein, 575 aa, Homo sapiens
MKRSLQALYCQLLSFLLILALTEALAFAIQEPSPRESLQVLPSGTPPGTMVTAPHSSTRHTSVVMLTPNPDGPPSQAAAPMATPTPRAEGHPPTHTISTIAATVTAPHSESSLSTGPAPAAMATTSSKPEGRPRGQAAPTILLTKPPGATSRPTTAPPRTTTRRPPRPPGSSRKGAGNSSRPVPPAPGGHSRSKEGQRGRNPSSTPLGQKRPLGKIFQIYKGNFTGSVEPEPSTLTPRTPLWGYSSSPQPQTVAATTVPSNTSWAPTTTSLGPAKDKPGLRRAAQGGGSTFTSQGGTPDATAASGAPVSPQAAPVPSQRPHHGDPQDGPSHSDSWLTVTPGTSRPLSTSSGVFTAATGPTPAAFDTSVSAPSQGIPQGASTTPQAPTHPSRVSESTISGAKEETVATLTMTDRVPSPLSTVVSTATGNFLNRLVPAGTWKPGTAGNISHVAEGDKPQHRATICLSKMDIAWVILAISVPISSCSVLLTVCCMKRKKKTANPENNLSYWNNTITMDYFNRHAVELPREIQSLETSEDQLSEPRSPANGDYRDTGMVLVNPFCQETLFVGNDQVSEI

SEQ ID NO: 3 - protein, 354 aa, Homo sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

(19) United States
(12) Patent Application Publication
(10) Pub. No.: US 2012/0128632 A1
(43) Pub. Date: May 24, 2012

(54) IDENTITY MARKERS

(76) Inventors: Jeffrey Keeler Teumer, Brookline, MA (US); Vladimir Mastyugin, Lexington, MA (US); Jizeng Qiao, Lexington, MA (US); Agatha Zawadzka, Essex, MA (US)

(21) Appl. No.: 13/386,456

(22) PCT Filed: Jul. 22, 2010
(86) PCT No.: PCT/US2010/042890

§ 371 Date: Jan. 23, 2012

Related U.S. Application Data
(60) Provisional application No. 61/228,003, filed on Jul. 23, 2009.

(51) Int. Cl. A61K 35/36; A61M 5/00; A61P 17/14; G01N 33/567; C12N 5/02

(52) U.S. Cl. 424/93.3; 435/7.21; 435/347; 435/383; 604/93.01

(57) ABSTRACT

Methods for identifying trichogenic dermal cells, including dermal papilla cells and dermal sheath cells, capable of inducing hair follicle formation when injected into skin are provided. It has been discovered that EGF latrophilin and seven transmembrane domain-containing protein 1 (ELTD1). Transmembrane Protein 108 (TMEM1 08), Hyaluronan and proteoglycan link protein 1 (HAPLN1) are biomarkers that can be used to detect, identify, and distinguish trichogenic dermal cells, i.e., that are able to induce hair follicle formation, from other skin cells. Populations of skin cells enriched with trichogenic dermal cells can be produced by selecting for and enriching for dermal cells that express ELTD1, TMEM1 08, HAPLN1, or a combination thereof.

### CROSS REFERENCE TO RELATED APPLICATIONS

This application claims benefit of U.S. Provisional Application No. 61/228,003, filed Jul. 23, 2009, which is hereby incorporated herein by reference in its entirety.

### FIELD OF THE INVENTION

The invention is generally related to the field of hair transplantation, more particularly to biomarkers and methods for the identification and/or isolation of trichogenic dermal cells, such as dermal papilla (DP) cells and dermal sheath (DS) cells.

### BACKGROUND OF THE INVENTION

Hair loss or alopecia is a common problem in both males and females regardless of their age. There are several types of hair loss, such as androgenetic alopecia, alopecia areata, telogen effluvium, hair loss due to systemic medical problems, e.g., thyroid disease, adverse drug effects and nutritional deficiency states as well as hair loss due to scalp or hair trauma, discoid lupus erythematosus, lichen planus and structural shaft abnormalities. (Hogan and Chamberlain, South Med J, 93(7):657-62 (2000)). Androgenetic alopecia is the most common cause of hair loss, affecting about 50% of individuals who have a strong family history of hair loss. Androgenetic alopecia is caused by three interdependent factors: male hormone dihydrotestosterone (DHT), genetic disposition and advancing age. DHT causes hair follicles to degrade and further shrink in size, resulting in weak hairs. DHT also shortens the anagen phase of the hair follicle growing cycle. Over time, more hairs are shed and hairs become thinner.

Possible options for the treatment of alopecia include hair prosthesis, surgery and topical/oral medications. (Hogan & Chamberlain, 2000; Bertolino, J Dermatol, 20(10):604-10 (1993)). While drugs such as minoxidil, finasteride and dutasteride represent significant advances in the management of male pattern hair loss, the fact that their action is temporary and the hairs are lost after stopping therapy continues to be a major limitation (Bouhanna, Dermatol Surg, 28:136-42 (2002); Avram, et al., Dermatol Surg, 28:894-900 (2002)). In view of this, surgical hair restoration and tissue engineering may be the only permanent methods of treating pattern baldness. The results from surgical hair transplantation can vary and early punch techniques often resulted in a highly unnatural “doll hair look” or “paddy field look” over the recipient area. Although advances have been made in surgical hair transplantation, for example, using single follicle hair grafts with 1 mm punches, the procedures are time consuming and costly and most important, the number of donor follicles on a given patient is limited.

Tissue engineering to treat hair loss includes transplanting cells into an area to induce hair follicle formation and subsequent hair shaft formation. Theoretically, this simple but effective method of tissue engineering may be employed to treat hair loss due to a variety of diseases, syndromes, and injuries and may provide significant insights into tissue and organ engineering. Hair follicle induction and growth involves active and continuous epithelial and mesenchymal interactions (Stenn & Paus, Physiol Reviews, 81:449-494, (2001)). In the embryo, the first hair follicles grow from a thickening of the primitive epidermis by signals arising from dermal cells. Early studies (Cohen, J Embryol Exp Morphol, 9:117-127 (1961)) using adult rodent hair follicles showed that the dissected deep mesenchymal portion of the hair follicle, the follicular or dermal papilla, when implanted under adult epidermis, will induce new hair follicles. This powerful inductive property is ascribed to a unique property of the cells in the papilla and about the base of the follicle—the dermal sheath (McElwee et al., J Invest Dermatol, 121:1267-1275 (2003)). Dermal papilla (DP) cells and dermal sheath (DS) cells from adult hair follicles can therefore be used to regenerate new hair follicles, i.e., are trichogenic dermal cells. Later work by Jahoda et al. (1984, Nature 311: 560-562) demonstrated that cultured DP cells can also induce hair follicle formation, raising the possibility that cultured DP cells and/or cultured DS cells could be used for hair regeneration or restoration in the cosmetic or therapeutic treatment of androgenetic alopecia and other hair loss disorders.

In order to be effective for hair regeneration, for example in a clinical setting, cultures of DP cells and/or DS cells should be relatively pure and free of contaminating cells. Achieving sufficiently pure cultures has been difficult because potentially contaminating cells such as keratinocytes and fibroblasts are abundant in scalp tissue. Fibroblasts in particular are difficult to distinguish from DP cells and DS cells using known methods because of the similarity in the appearance and growth characteristics of these cell types.

Currently, methods which endeavour to achieve sufficiently pure DP cell and/or DS cell cultures focus on the initial isolation of cells, from scalp or other tissues, prior to culturing. One prior art method to isolate DP cells and/or DS cells relies on microdissection, in which DP are manually separated from the surrounding hair follicle and skin tissue using fine tools manipulated under microscopic observation (Jahoda & Oliver, 1981, Br J Dermatol 105: 623-627). This method is relatively time-consuming and labor-intensive, and it requires physical separation of the DP and/or DS from the surrounding tissue to remove contaminating cell types in the skin, such as keratinocytes and fibroblasts. Other prior art methods employ physical and/or enzymatic techniques to isolate DP cells and/or DS cells (see Chiu et al., 1993, J Formos Med Assoc 92: 1029-1033; Warren et al., 1992, J Invest Dermatol 98: 693-699; and Wu et al., 2005, Arch Dermatol Res 297: 60-67), but these methods are also relatively time-consuming and labor-intensive, and do not provide an efficient means to selectively purify the desired cells away from contaminants.

Therefore, it is an object of the invention to provide biomarkers for identifying and enriching dermal cells, such as DP cells and DS cells, that are capable of inducing hair follicle formation (i.e., trichogenic dermal cells).

It is another object of the invention to provide cell populations enriched with trichogenic dermal cells, such as DP cells and DS cells.

It is another object of the invention to provide methods and compositions for treating hair loss in a subject.

### SUMMARY OF THE INVENTION

Methods for identifying dermal cells capable of inducing hair follicle formation when injected into skin are provided. It has been discovered that the membrane proteins ELTD1 and TMEM108 are biomarkers that can be used to detect, identify, and distinguish dermal papilla (DP) cells and/or dermal sheath (DS) cells from other skin cells. It has also been discovered that the extracellular matrix molecule HAPLN1 is associated with the DP cells and/or DS cells in a manner sufficient to detect, identify, and distinguish DP and/or DS cells from other skin cells. Populations of cells enriched for DP and/or DS cells can therefore be produced by selecting for and enriching for skin cells that express ELTD1 or TMEM108, or that are associated with HAPLN1.

These methods provided for the isolation and/or purification of DP cells and/or DS cells away from contaminating cell types and/or tissue debris in a manner that greatly reduces the need for operator intervention. This reduces the time and labor required to start DP cell and/or DS cell cultures. Therefore, methods of producing an enriched population of trichogenic dermal cells, such as DP cells and/or DS cells, are provided.

A population of cells enriched for trichogenic dermal cells, such as DS cells and/or DP cells, is also provided. The cells can have from about 10 percent ELTD1+ dermal cells to about 100 percent ELTD1+ cells. The cells can have from about 10 percent TMEM108+ dermal cells to about 100 percent TMEM108+ cells. The cells can have from about 10 percent HAPLN1+ dermal cells to about 100 percent HAPLN1+ cells.

Skin cell populations are also provided that contain an enriched population of trichogenic dermal cells combined with epidermal cells for use in inducing hair follicle formation.

Methods for inducing hair follicle formation are also provided. These methods can involve administering to a subject a population of trichogenic dermal cells enriched for ELTD1 or TMEM108 expression, or association with HAPLN1.

### BRIEF DESCRIPTION OF THE DRAWINGS

FIG. 1 is a schematic view of methodology for immunomagnetic isolation of Dermal Papilla (DP) cells using anti-HAPLN1 antibodies.

### DETAILED DESCRIPTION OF THE INVENTION

### I. Definitions

To facilitate understanding of the disclosure, the following definitions are provided:

The term “trichogenic cells” refers to skin cells that induce hair follicle formation. Induction of hair follicles can be direct or indirect.

The term “skin” refers to the outer covering of an animal. In general, the skin includes the epidermis and the dermis. Skin cells can include cells in or around a hair follicle, including fibroblasts, keratinocytes, melanocytes, dermal papilla cells, dermal sheath cells, and outer root sheath cells.

The term “trichogenic dermal cells” refers to dermal cells, such as dermal papilla (DP) cells and dermal sheath (DS) cells, that induce hair follicle formation.

The term “effective amount” refers to an amount of cells needed to induce hair follicle formation.

The terms “individual”, “host”, “subject”, and “patient” are used interchangeably herein, and refer to a mammal, including, but not limited to, murines, simians, humans, mammalian farm animals, mammalian sport animals, and mammalian pets.

The term “biomarker” refers to a nucleic acid or protein whose expression or presence is indicative of DP cells or DS cells. Representative biomarkers, include, but are not limited to EGF latrophilin and seven transmembrane domain-containing protein 1 (ELTD1), Transmembrane Protein 108 (TMEM108), and Hyaluronan and proteoglycan link protein 1 (HAPLN1).

The term “enriched” refers to a population of cells having an increase in the percentage of a given cell relative to reference skin cell populations. For example, as used herein, “enriched trichogenic dermal cells” refers to a population of cells that contains at least 30%, 40%, 50%, 60%, 70%, 80%, 90%, 95%, 97%, 98%, 99% or 100% DP cells and/or DS cells.

The term “isolated” refers to cells that are in an environment different from that in which the cells naturally occur e.g., separated from its natural milieu such as by separating dermal cells from a hair follicle.

### II. Enriched Dermal Papilla Cells and Dermal Sheath Cells

A. Biomarkers for DP cells and DS cells

Biomarkers are provided that are differentially expressed on or by trichogenic dermal cells, such as DP cells and/or DS cells. Therefore, these biomarkers are not detectable on or by other skin cells, such as fibroblasts or keratinocytes. Therefore, isolated populations of DP cells and/or DS cells may be obtained using the disclosed biomarkers in combination with a variety of isolation methods known to skilled artisans.

The biomarker of the disclosed methods can be EGF latrophilin and seven transmembrane domain-containing protein 1 (ELTD1), Transmembrane Protein 108 (TMEM108), or Hyaluronan and proteoglycan link protein 1 (HAPLN1).

ELTD1 (as known as ETL, KPG_{—}003 and hCG_{—}14667 or gene products thereof) is a membrane protein potentially involved in dicarboxylic acid transport. Human ELTD1 has the following amino acid sequence:

(SEQ ID NO: 1)
MKRLPLLVVF STLLNCSYTQ NCTKTPCLPN AKCEIRNGIE
ACYCNMGFSG NGVTICEDDN ECGNLTQSCG ENANCTNTEG
SYYCMCVPGF RSSSNQDRFI TNDGTVCIEN VNANCHLDNV
CIAANINKTL TKIRSIKEPV ALLQEVYRNS VTDLSPTDII
TYIEILAESS SLLGYKNNTI SAKDTLSNST LTEFVKTVNN
FVQRDTFVVW DKLSVNHRRT HLTKLMHTVE QATLRISQSF
QKTTEFDTNS TDIALKVFFF DSYNMKHIHP HMNMDGDYIN
IFPKRKAAYD SNGNVAVAFV YYKSIGPLLS SSDNFLLKPQ
NYDNSEEEER VISSVISVSM SSNPPTLYEL EKITFTLSHR
KVTDRYRSLC AFWNYSPDTM NGSWSSEGCE LTYSNETHTS
CRCNHLTHFA ILMSSGPSIG IKDYNILTRI TQLGIIISLI
CLAICIFTFW FFSEIQSTRT TIHKNLCCSL FLAELVFLVG
INTNTNKLFC SIIAGLLHYF FLAAFAWMCI EGIHLYLIVV
GVIYNKGFLH KNFYIFGYLS PAVVVGFSAA LGYRYYGTTK
VCWLSTENNF IWSFIGPACL IILVNLLAFG VIIYKVFRHT
AGLKPEVSCF ENIRSCARGA LALLFLLGTT WIFGVLHVVH
ASVVTAYLFT VSNAFQGMFI FLFLCVLSRK IQEEYYRLFK
NVPCCFGCLR.

TMEM108 (also known as MGC3040 or a gene product thereof) is a membrane protein potentially involved in potassium ion transport. Human TMEM108 has the following amino acid sequence:

(SEQ ID NO: 2)
MKRSLQALYC QLLSFLLILA LTEALAFAIQ EPSPRESLQV
LPSGTPPGTM VTAPHSSTRH TSVVMLTPNP DGPPSQAAAP
MATPTPRAEG HPPTHTISTI AATVTAPHSE SSLSTGPAPA
AMATTSSKPE GRPRGQAAPT ILLTKPPGAT SRPTTAPPRT
TTRRPPRPPG SSRKGAGNSS RPVPPAPGGH SRSKEGQRGR
NPSSTPLGQK RPLGKIFQIY KGNFTGSVEP EPSTLTPRTP
LWGYSSSPQP QTVAATTVPS NTSWAPTTTS LGPAKDKPGL
RRAAQGGGST FTSQGGTPDA TAASGAPVSP QAAPVPSQRP
HHGDPQDGPS HSDSWLTVTP GTSRPLSTSS GVFTAATGPT
PAAFDTSVSA PSQGIPQGAS TTPQAPTHPS RVSESTISGA
KEETVATLTM TDRVPSPLST VVSTATGNFL NRLVPAGTWK
PGTAGNISHV AEGDKPQHRA TICLSKMDIA WVILAISVPI
SSCSVLLTVC CMKRKKKTAN PENNLSYWNN TITMDYFNRH
AVELPREIQS LETSEDQLSE PRSPANGDYR DTGMVLVNPF
CQETLFVGND QVSEI.

Neither ELTD1 nor TMEM108 have previously been reported to be associated specifically with DP cells and/or DS cells among dermal cells.

HAPLN1 (“Hyaluronan and proteoglycan link protein 1”) is an extracellular matrix molecule not directly attached to the cell. Thus, although this marker protein may be considered as being only indirectly associated with DP and/or DS cells, it nonetheless can be used through conventional methodologies to detect and purify DP cells and/or DS cells. HAPLN1 is an extracellular matrix protein found in cartilage. The protein is reported in the prior art to stabilise aggregates of hyaluronic acid and proteoglycans, including aggrecan and inter-alpha-trypsin inhibitor, and is involved in cell to cell adhesion. HAPLN1 has not previously reported to be associated specifically with DP cells and/or DS cells among dermal cells. Human HAPLN1, also known as CRTL1 (or a gene product thereof), has the following amino acid sequence:

(SEQ ID NO: 3)
MKSLLLLVLI SICWADHLSD NYTLDHDRAI HIQAENGPHL
LVEAEQAKVF SHRGGNVTLP CKFYRDPTAF GSGIHKIRIK
WTKLTSDYLK EVDVFVSMGY HKKTYGGYQG RVFLKGGSDS
DASLVITDLT LEDYGRYKCE VIEGLEDDTV VVALDLQGVV
FPYFPRLGRY NLNFHEAQQA CLDQDAVIAS FDQLYDAWRG
GLDWCNAGWL SDGSVQYPIT KPREPCGGQN TVPGVRNYGF
WDKDKSRYDV FCFTSNFNGR FYYLIHPTKL TYDEAVQACL
NDGAQIAKVG QIFAAWKILG YDRCDAGWLA DGSVRYPISR
PRRRCSPTEA AVRFVGFPDK KHKLYGVYCF RAYN.

In preferred embodiments, trichogenic dermal cells, such as DP cells and DS cells, can be detected, identified, and enriched in assays that detect protein expression. Trichogenic dermal cells, such as DP cells and DS cells, can be detected, identified, and enriched using a variety of conventional techniques including, but not limited to immunological, spectrophotometric, fluorometric, and colormetric assays.

In a preferred embodiment, trichogenic dermal cells, such as DP cells and DS cells, are detected using antibodies that specifically bind the one or more biomarkers disclosed herein. ELTD1 antibodies are commercially available, for example, from Imgenex (San Diego, Calif.) and LifeSpan BioSciences (Seattle, Wash.). TMEM108 antibodies are commercially available, for example, from Proteintech Group (Chicago, Ill.). HAPLN1 antibodies are commercially available, for example, from R&D Systems (Minneapolis, Minn.), Novus Biologicals (Littleton, Colo.), and Santa Cruz Biotechnology (Santa Cruz, Calif.). The antibody can be labelled with a detectable label such as fluorescent labels, chemiluminescent labels, chromophores, antibodies, enzymatic markers, radioactive isotopes, affinity tags and photoreactive groups.

B. Cell Populations Enriched for DP Cells and DS Cells

Populations of skin cells enriched for trichogenic dermal cells, such as DP cells and DS cells, are also provided. The population of skin can be enriched for cells expressing the one or more biomarkers disclosed herein by at least about 5%, 10%, 15%, 20%, 25%, 30%, 35%, or 40%. The initial skin cell population can be obtained from a mammalian subject, preferably from a human. The initial skin cell population can be heterogeneous in that it can contain, for example, DP cells, DS cells, fibroblasts, melanocytes, and keratinocytes. In a preferred embodiment, the enriched trichogenic dermal cell population is homogeneous in that it contains only trichogenic dermal cells, such as DP cells and DS cells.

Methods for enriching cell populations based on protein expression on (or associated with) the cell surface are known in the art and include, but are not limited to, flow cytometry and immunological separation techniques. A preferred technique for enriching DP cells and DS cells uses commercially available reagents such as CELLection™ Biotin Binder Kit from Invitrogen. Generally, biotinylated antibodies to the one or more disclosed biomarkers are added to a suspension of skin cells. Next, streptavidin conjugated beads are added to the suspension and bind to biotinylated antibody bound to cells positive for the one or more biomarkers. A magnet is then used to separate the DP cells and/or DS cells from the other skin cells and thereby form two populations of cells. One population is enriched with DP cells and/or DS cells and the other population has a significantly reduced number of DP cells and/or DS cells.

A skin cell population of enriched trichogenic dermal cells, such as DP cells and DS cells, combined with epidermal cells is also provided. The epidermal:dermal can be present in the suspension in a ratio of about 0:1, 1:1, 1:2 and 1:10. The suspension of trichogenic dermal cells and epidermal cells can further include additional cell types, such as melanocytes.

Aggregates of enriched trichogenic dermal cells and epidermal cells are also provided. The epidermal:dermal can be present in the aggregates in a ratio of about 0:1, 1:1, 1:2 and 1:10. The aggregate of trichogenic dermal cells and epidermal cells can further include additional cell types, such as melanocytes. The cells can be aggregated by suspension growth in a non-stick tissue culture dish, or by centrifugation of the cultured cells. In certain embodiments, a suitable aggregation enhancing substance may be added to the cells prior to, or at the time of, implantation. Suitable aggregation enhancing substances include, but are not limited to, glycoproteins such as fibronection or glycosaminoglycans, dermatan sulfate, chondroitan sulfates, proteoglycans, heparin sulfate and collagen.

C. Kits

Kits are provided that include a container containing enriched trichogenic dermal cells, such as DP cells and/or DS cells. These kits can further contain, for example, one or more reagents, such as culture medium, for culture of the trichogenic dermal cells. These kits can contain endodermal cells for co-culture or co-transplantation with the dermal cells. These kits can contain suitable aggregation enhancing substances, including but not limited to, glycoproteins such as fibronection or glycosaminoglycans, dermatan sulfate, chondroitan sulfates, proteoglycans, heparin sulfate and collagen.

Kits are also provided that include a container containing antibodies that selectively bind the one or more biomarkers disclosed herein for enrichment of trichogenic dermal cells, such as DP cells and/or DS cells.

### III. Methods of Identifying and Purifying DP and/or DS Cells

The one or more biomarkers disclosed herein can be used to identify trichogenic dermal cells, such as DP cells and/or DS cells. Generally, cells are harvested from an animal, for example a mouse or human. The cells can be autologous or allogenic. Tissue, preferably scalp tissue, is obtained from a subject, such as a human fetus, child, or adult, and processed to obtain dissociated cells using techniques known in the art. The cells are a mixed population of cells containing DP and/or DS cells and other skin cells, such as fibroblasts and keratinocytes. In some embodiments the mixed population of cells includes both dermal and epidermal cells. The dermal and epidermal cells can be trichgenic or non-trichogenic or a combination thereof.

Trichogenic dermal cells, such as DP and/or DS cells, in a mixed population of skin cells are identified by assaying the cells for expression of, or association with, one or more biomarkers disclosed herein. For example, the biomarker can be ELTD1, TMEM108, HAPLN1, or a combination thereof.

In one embodiment a population of cells enriched for expression of one or more trichogenic biomarkers is obtained by cell sorting using CELLection™ Biotin Binder Kit. Both direct and indirect methods can be employed. Basically, a biotinylated anti-biomarker antibody is added to the cell sample at 1 μg per 1 million cells (indirect method) or added to streptavidin coated beads at 2 μg/25 ul beads (direct method) and incubated at 4° C. overnight. The streptavidin coated beads can be moved using a magnet. Next, the streptavidin coated beads and cell sample are mixed together so the biomarker positive cells attach to the streptavidin coated beads through the biotinylated anti-biomarker antibody. The bead-bound-cells are then separated from other cells by a magnet. The biomarker positive cells are then released from the magnetic beads. The beads are then removed using magnets.

In another embodiment, biomarker expression is detected by Guava Analyzer. Briefly, cells are first incubated with a Phycoerythrin conjugated anti-biomarker antibody at 4° C. for half an hour. Then the cells are washed two times with Dulbecco's Phosphate Buffered Saline (DPBS) with bovine serum albumin (0.1% BSA) plus antibiotic (clindamycin, actinomycin, streptomycin). Biomarker expression level is measured by GUAVA Analyzer.

The method can in some embodiments, involve detection in the cell of the nucleic acid encoding ELTD1 or TMEM108. Methods for identifying nucleic acid or protein biomarkers are known in the art. Quantitative Real-Time PCR, flow cytometry and immunological techniques are preferred.

### IV. Methods of Using Enriched DP and DS Cells

Populations of skin enriched for trichogenic dermal cells, such as DP cells and/or DS cells, can be used to replace, augment, or restore hair. Enriched trichogenic dermal cells can be injected subcutaneously or intradermally to induce the formation of new hair follicles. The new hair follicles generate new hair shafts. Thus, the enriched trichogenic dermal cells can replace or augment existing hair follicles by inducing the formation of new or additional hair follicles that generate new hair shafts. Alternatively, the populations of enriched trichogenic dermal cells, such as DP cells and/or DS cells, can be injected subcutaneously or intradermally to induce existing hair follicles to generate new terminal hair. For example, a population of enriched trichogenic dermal cells can be injected adjacent to one or more existing hair follicles that produce vellus hair. The enriched trichogenic dermal cells then induce the vellus hair follicle to produce terminal hair. These methods for using enriched trichogenic dermal cell populations are described in more detail below.

A. Hair Follicle Induction

Enriched trichogenic dermal cells, such as DP cells and/or DS cells, can be used to generate new hair follicles in a subject. Typically, the enriched dermal cell population is autologous or allogenic.

Subjects to be transplanted with enriched trichogenic dermal cells, such as DP cells and/or DS cells, include any subject that has an insufficient amount of hair or an insufficient rate of hair growth at a site or region of skin. The enriched trichogenic dermal cells can be used to treat hair loss resulting from androgenetic alopecia, wounding, trauma, scarring, telogen effluvium, genetic pattern baldness or with hormonal disorders that decrease hair growth or cause loss of hair. Subjects may have these conditions or be at risk for the development of these conditions, based on genetic, behavioral or environmental predispositions or other factors. Other suitable subjects include those that have received a treatment, such as chemotherapy or radiation, that causes a decrease in hair growth or a loss of hair. The enriched trichogenic dermal cells can also be used to treat scalp or hair trauma, structural hair shaft abnormalities, or a surgical procedure, such as a skin graft, which results in an area of skin in need of hair follicles.

In certain embodiments, the enriched population of trichogenic dermal cells, such as DP cells and/or DS cells, is combined with epidermal cells prior to implantation in a subject. Preferred locations for implantation include body skin including, but not limited to the subject's scalp or face. In one embodiment enriched trichogenic dermal cells are injected alone.

In a preferred embodiment, enriched trichogenic dermal cells and epidermal cells are cultured and expanded prior to implantation to obtain a sufficiently large number of cells suitable for implantation at multiple sites of a host to form new hair follicles. The cells are cultured in a manner that maintains the trichogenic activity of the dermal cells. Methods for culturing dissociated dermal and epidermal cells are known in the art. Dermal cells may be cultured separately from epidermal cells or may be co-cultured with epidermal cells. Exemplary methods for culturing dermal cells are provided in Roh, et al., Physiol. Genomics, 19:207-17 (2004) and McElwee, et al., Jour. Invest. Dermatol., 121(6):1267-75 (2003).

Suitable cell culture media include commercially available media, such as Dulbecco's Modified Eagle Medium/Nutrient Mixture F-12 (DMEM/F-12), RPMI-1640 and Ham's F10 (Sigma). The medium may be supplemented as appropriate with serum (such as fetal bovine serum, calf serum or horse serum), hormones or other growth factors (such as insulin, epidermal growth factor, Wnt polypeptides, or transferrin), ions (such as sodium, chloride or calcium), buffers (such as HEPES), nucleosides or trace elements.

The cells that are implanted into the subject may be autologous, allogenic or xenogenic. In one embodiment, enriched trichogenic dermal cells, such as DP cells and/or DS cells, and epidermal cells are obtained from skin sections from a single allogenic donor. In another embodiment, trichogenic dermal cells and epidermal cells are obtained from skin sections from more than one donor. For example, enriched trichogenic dermal cells may be derived from one donor and epidermal cells from another donor. In a preferred embodiment, the cells that are implanted are autologous.

Enriched trichogenic dermal cells and epidermal cells can be combined at an appropriate ratio prior to implanting into the subject. The epidermal:dermal ratio would range from 0:1, 1:1, 1:2 and 1:10. Dermal cells and epidermal cells can be further combined with additional cell types, such as melanocytes, prior to implantation. The enriched trichogenic dermal cells and epidermal cells to be implanted can be subjected to physical and/or biochemical aggregation prior to implanting to induce and/or maintain aggregation of the cells within the transplantation site. For example, the cells can be aggregated by suspension growth in a non-stick tissue culture dish, or by centrifugation of the cultured cells. In certain embodiments, a suitable aggregation enhancing substance may be added to the cells prior to, or at the time of, implantation. Suitable aggregation enhancing substances include, but are not limited to, glycoproteins such as fibronection or glycosaminoglycans, dermatan sulfate, chondroitan sulfates, proteoglycans, heparin sulfate and collagen.

The enriched trichogenic dermal cells, such as DP cells and/or DS cells, may be implanted into a subject using routine methods known in the art. Various routes of administration and various sites can be used. For example, the cells can be introduced directly between the dermis and the epidermis of the outer skin layer at a treatment site. This can be achieved by raising a blister on the skin at the treatment site and introducing the cells into fluid of the blister. The cells may also be introduced into a suitable incision extending through the epidermis down into the dermis. The incision can be made using routine techniques, for example, using a scalpel or hypodermic needle. The incision may be filled with cells generally up to a level in direct proximity to the epidermis at either side of the incision. In a preferred embodiment, the cells are delivered using a device as described in US Patent Application Publication No. 2007/0233038 to Pruitt, et al.

The dosage of cells to be injected is typically between about one million to about four million cells per square cm.

In another embodiment, a plurality of small recipient sites, for example, 10, 50, 100, 500 or 1000 or more is formed in the skin into which the cells are transplanted. Each perforation can be filled with a plurality of cells. The size and depth of the perforations can be varied. The perforations in the skin can be formed by routine techniques and can include the use of a skin-cutting instrument, e.g., a scalpel or a hypodermic needle or a laser (e.g., a low power laser). Alternatively, a multiple-perforation apparatus can be used having a plurality of spaced cutting edges formed and arranged for simultaneously forming a plurality of spaced perforations in the skin. The cells can be introduced simultaneously into a plurality of perforations in the skin.

The number of cells introduced into each perforation can vary depending on various factors, for example, the size and depth of the opening and the overall viability and trichogenic activity of the cells. In one embodiment about 50,000 to about 2,000,000 cells are delivered per injection. The cell concentration can be about 5,000 to about 1,000,000 cells/μl, typically about 50,000 cells/μl to about 75,000 cells/μl. A representative volume of cells delivered per injection is about 1 to about 10 μl, preferably about 4 μl. In one embodiment, 1 to 100 injections per cm², typically 1 to 30 injections per cm² are made in the skin, preferably the scalp.

The epidermal cells, dermal cells, or combinations thereof may be combined with a pharmacologically and/or physiologically suitable carrier such as saline solution, phosphate buffered saline solution, Dulbecco's Phosphate Buffered Saline (“DPBS”), DMEM, D-MEM-F-12 or HYPOTHERMOSOL-FRS from BioLifeSolutions (Bothell, Wash.) or a preservation solution such as a solution including, but not limited to, distilled water or deionized water, mixed with potassium lactobionate, potassium phosphate, raffinose, adenosine, allopurinol, pentastarch prostaglandin El, nitroglycerin, and/or N-acetylcysteine into the solution. Typically, the injected cells are suspended in cell culture media used to culture the cells. The preservation solution employed may be similar to standard organ and biological tissue preservation aqueous cold storage solutions such as HYPOTHERMOSOL-FRS from BioLifeSolutions (Bothell, Wash.).

The cells and the carrier may be combined to form a suspension suitable for injection. Each injection will typically include about 1.0 μl to about 10 μl of composition or suspension. The injection may be performed with any suitable needle, syringe or other instrument. A 25 gauge needle attached to a syringe loaded with the composition or suspension may be used. Alternatively, a hubless insulin syringe may also be used to inject the composition into skin of a mammal. The suspension may also be delivered by other suitable methods, such as spreading the composition or suspension over superficial cuts of the skin or pipetting the composition or suspension into an artificially created wound.

The use of dermal and/or epidermal cells derived from an allogenic source may require administration of an immunosuppressant, alteration of histocompatibility antigens, or use of a barrier device to prevent rejection of the implanted cells. Cells can be administered alone or in conjunction with a barrier or agent for inhibiting or reducing immune responses against the transplanted cells in a recipient subject. For example, an immunosuppressive agent can be administered to a subject to inhibit or interfere with normal response in the subject. The immunosuppressive agent can be an immunosuppressive drug that inhibits T cell/or B cell activity in the subject or an antibody to t-cells. Suitable immunosuppressive drugs are commercially available. An immunosuppressive agent can be administered to a subject at a dosage sufficient to achieve the desired therapeutic effect (e.g., inhibition of rejection of the cells).

In some embodiments, the subject is treated, topically and/or systematically, with a hair growth promoting substance before, at the same time as, and/or after the transplantation of cells to enhance hair growth. Suitable hair growth promoting substances can include, e.g., minoxidil, cyclosporin, and natural or synthetic steroid hormones and their enhancers and antagonists, e.g., anti-androgens, all of which are commercially available.

B. Terminal Hair Induction

Another embodiment provides a method for inducing vellus hair to become terminal hair. Vellus hair is the fine, non-pigmented hair (peach fuzz) that covers the body of children and adults. Terminal hair is developed hair, which is generally longer, coarser, thicker and darker than the shorter and finer vellus hair. The growth of vellus hair is not affected by hormones; whereas, the growth of terminal hair is affected by hormones. Vellus hair is also present in male pattern baldness.

In one embodiment a population of skin cells enriched for trichogenic dermal cells, such as DP and/or DS cells, are injected into a skin as described above. The enriched trichogenic dermal cells are obtained as described above and are typically autologous or allogenic cells. The cells are injected adjacent to vellus hair or vellus hair follicles. Multiple injections of enriched DP and/or DS cells can be delivered to an area of skin containing vellus hair to induce as many vellus hair follicles as possible to become terminal hair follicles. It will be appreciated that the number of injections and volume of cells to be injected can be routinely determined by one of skill in the art.

In another embodiment, enriched trichogenic dermal cells, such as DP cells and/or DS cells, are injected into skin in an amount effective to induce formation of hair follicles and to induce vellus hair follicles to become terminal hair follicles. In one embodiment, the number of cells injected is effective to induce hair follicle formation in a period of about two weeks to about twelve weeks. In another embodiment, the injected cells induce terminal hair formation from vellus hair in a period of about two weeks to about twelve weeks.

### EXAMPLES

### Example 1

### Microarray Analysis of Genes Expressed in DP/DS Cells and not in Fibroblasts or Keratinocytes

In order to identify genes that are expressed in DP/DS cells and not in fibroblasts or keratinocytes, gene expression was compared in these cell types using microarray analysis.

Materials and Methods

Microarray Screening Methodology

Total RNA was prepared from 9 cell culture samples and 3 freshly isolated tissue samples. The 12 samples fell into the groups below:

Group 1: cultured human dermal fibroblasts (HDF) from 3 independent donors;

Group 2: cultured human keratinocytes (HK) from 3 independent donors;

Group 3: cultured dermal papilla cells (DP cells) from 3 independent donors; and

Group 4: freshly isolated dermal papillae (DPfr) from 3 independent pools of donors.

RNA extraction, purification, analysis, labelling, profiling on microarrays and primary microarray data analysis was performed by ALMAC Diagnostics (Durham, N.C., USA) in accordance with Minimum Information About a Microarray Experiment (MIAME) standards (see Brazma et al., 2001, Nature Genetics 29: 365-371).

The 12 RNA samples were assessed for quality by spectrophotometry and Agilent Bioanalyzer analysis. High quality RNA samples were used to generate labelled nucleic acid samples that were profiled on Affymetrix Human Genome U133 Plus2 Arrays. Nucleic acid preparations were amplified using the NuGEN™ Ovation™ RNA Amplification System V2. The amplified cDNA was then labeled using the FL-Ovation™ cDNA Biotin Module V2.

The resultant labelled cDNA was hybridised onto Affymetrix GeneChip® arrays. Following the hybridisation, the array was washed and stained using a GeneChip® Fluidics Station 450 using the appropriate fluidics script, before being inserted into the Affymetrix autoloader carousel and scanned using the GeneChip® Scanner 3000.

Rosetta Resolver Gene Expression Analysis system was used for microarray data analysis. Data quality control included Data Distribution Plot analysis; Hierarchical Clustering; and Data Reduction Analysis with Principal Components Analysis (PCA) applied to the data to produce a set of expression patterns known as principal components. No outliers were detected and all 12 samples were used in the data analyses.

Data Analysis

Statistical analysis (ANOVA) with multiple testing correction (FDR adjusted P*-value<0.001 and post hoc p-value<0.001) were used to generate a “stringent gene list” for the three post hoc comparisons (DP cells vs. DPfr, HDF, and HK, respectively) based on less stringent genes which passed filters of background correction and 3× standard deviations (>7.94) and ratio error p-value<0.01.

Candidate Validation

108 candidates from the stringent gene list were selected for further validation based on the relative levels of gene expression profiled below:

1. DPfr>DP cells>(HDF and HK)

2. DP cells>DPfr>(HDF and HK).

This further validation was performed by QPCR using standard methods. Validation was performed on both amplified and non-amplified RNA samples, and the results were very similar indicating that RNA amplification did not introduced significant variability in the samples.

Results

A total of 80 candidates from the stringent gene list fit the desired gene expression profile in 1 or 2 above. The 80 candidates were further screened according to their cellular location based on published information. Candidates that encoded proteins of which at least a portion was localized to the exterior of the cell were chosen for further analysis. These candidates included transmembrane proteins, external membrane associated proteins, and extracellular matrix proteins. Of the 80 candidates, 25 fit the extracellular location criterion.

These 25 extracellular proteins were subjected to a final validation step in which commercially available antibodies were used for assessing the protein expression profile to confirm their DP cell-specific expression. Immunostaining was performed on cultures of DP cells, HDF, and HK as well as sections of human scalp tissue.

In the following Examples, evidence is provided that three exemplary markers (HAPLN1, TMEM108, and ELTD1) are expressed in cultured DP cells and not in cultured HDF or HK, and in the dermal papillae and dermal sheathes but not in the dermis or epidermis in scalp tissue sections. One exemplar marker (HAPLN1) is used to isolate a whole DP from a suspension of isolated DP, while the other two exemplar markers (TMEM108 and ELTD1) are each used to isolate DP cells from a mixed skin cell suspension.

### Example 2

### HAPLN1 is Expressed Specifically in DP/DS Cells in Hair Follicles

Materials and Methods

Human hair follicles were isolated by microdissection from a human scalp biopsy, immersed in 2M sucrose solution for 48 h, embedded in TFS compound (Triangle Biomedical Research, Cat. No. H-TFM) blocks, sectioned at 8 μm intervals and placed onto Poly-L-Lysine-coated microscope slides (Polysciences, Inc., Cat. No. 22247). Frozen sections were fixed in 100% acetone for 15 min at −20° C. After two PBS washes, sections were treated with Peroxidase Blocking reagent (Dako, Cat. No. REFS201) for 10 min at room temperature, washed with PBS three times and treated with components of Avidin/Biotin Blocking Kit (Vector Laboratories, Cat. No. SP2001) according to manufacturer's protocol. Immunostaining was perfumed using Peroxidase Goat IgG Kit (VECTASTAIN ABC kit, Vector Laboratories, Cat. No. PK-4005) as described in manufacturer's protocol. Primary antibody used was polyclonal goat anti-human HAPLN1 (R&D Systems, Cat. No. AF2608), dilution 1:20. Immunostained sections were briefly counterstained with Hemotoxilin 2 (Richard Allan Scientific, Cat. No. 7231), washed and dehydrated in a series of 70, 95 and 100% 1 min ethanol washes and finally mounted with Clear-Rite 3 (Richard Allan Scientific, Cat. No. 6901). Stained sections were viewed under microscope.

Results

An immunostained section of scalp tissue was obtained from human scalp biopsies cut into small pieces with 7×7 mm base and treated as described using the same methodology. HAPLN1 was found to be preferentially expressed in DP and/or DS and not in other skin structures such as epidermis and dermis.

### Example 3

### HAPLN1 is Preferentially Expressed in Cultured DP Cells and/or DS Cells but not in Cultured HDF or in Cultured HK

Materials and Methods

HK and HDF were obtained from infant foreskin. DP and DS cells were obtained from adult human donors.

HDF were cultured in DMEM supplemented with 10% FBS, 2 mM L-Glutamine and 0.05 mg/ml Gentamicin. HK were cultured in EpiLife Medium supplemented with EDGS (Invitrogen). DP cells were cultured in Dermal Papilla Growth Medium (combination of equal parts of human keratinocyte condition medium and Chang Medium. All cells were cultured in 8-well microscope chamber slides (LabTek II Chamber Slide System, Nulge, Nunc International, Cat. No. 154534).

For immunostaining of the cultures, cells were fixed in a mixture of 5% acetic acid and 95% ethanol for 5 min at room temperature. Fixed cells were washed twice with PBS and immunostained with a Mouse Monoclonal Anti-human HAPLN1 antibody (R&D Systems, Cat. No. MAB2608, 10 μg/ml), using VECTASTAIN ABC Kit (Mouse IgG) (Vector Laboratories, Cat. No. PK-4002) as directed by manufacturer. After immunostaining, cells were counterstained with hematoxylin.

Results

HAPLN1 immunostaining was detected in cultured DP cells. All DP cells were seen to express HAPLN1. Cultured HDF and cultured HK, respectively, did not express HAPLN1.

### Example 4

### ELTD1 is Preferentially Expressed in Cultured DP/DS Cells but not in Cultured Human Dermal Fibroblasts or in Cultured Human Keratinocytes

Materials and Methods

Materials and methods were as for Example 3, except that polyclonal rabbit anti-human ETL (ELTD1) (Abeam, Cat. No. ab12989, dilution 1:100) and VECTASTAIN ABC Kit (Rabbit IgG) (Vector Laboratories, Cat. No. PK-4001) were used.

Results

ELTD1 was preferentially expressed in cultured human DP/DS cells.

### Example 5

### TMEM108 is Preferentially Expressed in Cultured DP/DS Cells but not in Cultured HDF or in Cultured HK

Materials and Methods

Materials and methods were as for Example 4, except that the antibody used for immunostaining TMEM108 was polyclonal rabbit anti-human TMEM108 (Abeam, Cat. No. ab49115, dilution 1:200)

Results

TMEM108 staining in cultured human DP/DS cells and showed that TMEM108 was expressed in these cells. In contrast, TMEM108 was not expressed in cultured HDF and cultured HK, respectively.

### Example 6

### Use of HAPLN1 for the Isolation of an Intact DP

Materials and Methods

Superparamagnetic beads used were Dynabeads® Sheep-anti Mouse IgG (4.5 μm diameter) (Invitrogen, Cat. No. SKU#110-31). The antibody was Mouse Monoclonal Anti-human HAPLN1 Antibody (R&D Systems, Cat. No. MAB2608).

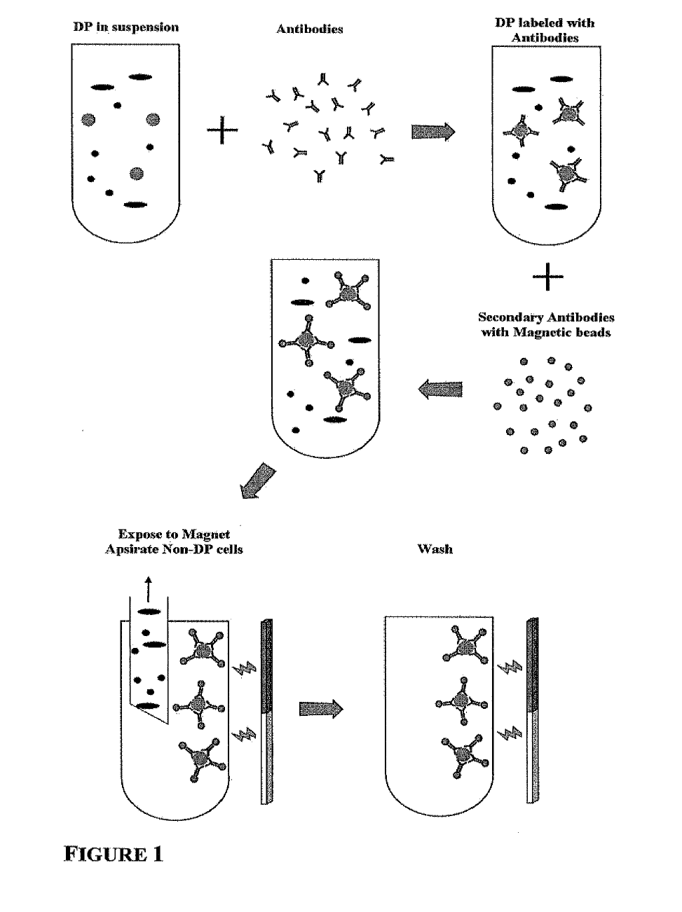

Whole human DP were microdissected from human scalp biopsy, placed into 1 ml of DMEM medium and labelled with 5 μg of Mouse Monoclonal Anti-human HAPLN1 for 30 min at room temperature under tilting and rotating. HAPLN1 Antibody-labelled DP were washed from unbound excess antibody and incubated with ˜1×10⁷ Dynabeads® Sheep-anti Mouse IgG in 1 ml of DMEM. Sheep-anti Mouse IgG recognises Mouse Monoclonal Anti-human HAPLN1 antibodies and thus the DP forms a complex with Dynabeads®. Finally, Dynabeads®-complexed DP were positively selected with a magnet. This isolation method is illustrated in FIG. 1. After rinsing to remove unbound contaminants, beaded DP were treated with 1 ml of 100 U/ml Collagenase Type 1 and 20 U/ml of DNase I in PBS (with Ca²⁺ and Mg²⁺) for 30 min at 37° C. The suspension was then transferred to a 10 cm culture dish.

Results

After rinsing the beaded DP as described above, the immunomagnetically-isolated DP were ready for plating.

### Example 7

### Use of ELTD1 to Isolate DP Cells from a Mixed Suspension Containing HDF and DP Cells

Materials and Methods

Dynabeads® M-280 Sheep anti-Rabbit IgG (2.8 μm diameter) (Invitrogen, Cat. No. 11203D) were used, together with polyclonal rabbit anti-human ETL (ELTD1) as in Example 4. Dyes used were Hoechst 33342® (Invitrogen, Cat. No. H3570) and Vibrant DiI® (Invitrogen, Cat. No. V22885).

HDF cells were obtained from infant foreskin. DP cells were obtained from adult human donors. Separate cultures of human DP cells and HDF were washed with PBS. The cells were detached from plastic culture dishes by incubation with 0.2 mg/ml ETDA solution for 10 min at 37° C. DP cells were counted and were then stained with Hoechst 33342® as directed by the manufacturer. HDF were counted and then stained with Vibrant DiI® dye according to the manufacturer's protocol. Stained cells were mixed in equal proportion to obtain a suspension of 1×10⁷ cells/ml and labelled with ELTD1 antibody (1 μg antibody per 10⁶ cells) in 1 ml of PBS supplemented with 0.1% of Bovine Serum Albumin (BSA) for 30 min at room temperature under rotating and tilting. Antibody-labeled cells were washed from unbound excess antibody and incubated with ˜5×10⁷ Dynabeads® Sheep-anti Rabbit in 0.5 ml of PBS supplemented with 0.1% of BSA at room temperature for 30 min under rotating and tilting. Sheep-anti Rabbit IgG recognizes Rabbit Polyclonal Anti-human Antibodies and thus the ELTD1 antigen-carrying cells form complexes with Dynabeads.

Dynabeads®-cell complexes were washed from unbound cells and subjected to differential counting under fluorescence microscopy with an appropriate filter.

Results

Differential counting of immunomagnetically-isolated cells revealed that the DP cell population represented 84% for ELTD1-positive counted cells in the DP-HDF mixture with 16% contaminating HDF. It is expected that further optimisation would result in higher levels of DP cell purity.

### Example 8

### Use of TMEM108 to Isolate DP Cells from a Mixed Suspension Containing HDF and DP Cells

Materials and Methods

Materials and methods were as described for Example 7, except that a polyclonal rabbit anti-human TMEM108 antibody (see Example 5) was used.

Results

Differential counting of immunomagnetically-isolated cells revealed that the DP cell population represented 88% for TMEM108-positive counted cells in DP-HDF mixture with 12% contaminating HDF. Again, it is expected that further optimisation would result in higher levels of DP cell purity. DP cells were stained with Hoechst 33342® and immunomagnetically-isolated with TMEM108 antibody from the DP-HDF mixture.

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A method for producing an enriched population of trichogenic dermal cells comprising
providing a heterogeneous skin cell population; and
selecting from the skin cell population cells expressing EGF latrophilin and seven transmembrane domain-containing protein 1 (ELTD1) or Transmembrane Protein 108 (TMEM108) on the cell surface, or selecting cells having Hyaluronan and proteoglycan link protein 1 (HAPLN1) associated with the cell surface, thereby forming a second population of cells enriched for trichogenic dermal cells.

2. The method of claim 1, wherein the trichogenic dermal cells are dermal papilla cells, dermal sheath cells, or a combination thereof.

3. The method of claim 1 wherein the trichogenic dermal cells are selected using antibodies to ELTD1, TMEM108, or HAPLN1 via fluorescence activated cell sorter or magnetic beads.

4. A population of dermal cells produced by the method of claim 1, comprising from about 10 percent ELTD1+ dermal cells to about 100 percent ELTD1+ cells.

5. A population of dermal cells produced by the method of claim 1, comprising from about 10 percent TMEM108+ dermal cells to about 100 percent TMEM108+ cells.

6. A population of dermal cells produced by the method of claim 1, comprising from about 10 percent HAPLN1+ dermal cells to about 100 percent HAPLN1+ cells.

7. A skin cell population comprising the population of dermal cells of claim 4 and epidermal cells.

8. The skin cell population of claim 7 wherein the epidermal cells are present in a ratio of epidermal to dermal cells effective to induce hair follicle formation when administered to a subject.

9. The skin cell population of claim 7 in a cell culture or shipping container.

10. The skin cell population of claim 7 in a device for injecting the cells into openings in the skin.

11. The skin cell population of claim 7, wherein the dermal cells are human dermal cells.

12. The skin cell population of claim 11, wherein the epidermal cells are human epidermal cells.

13. A method for inducing hair follicle formation in a subject comprising administering to the subject an effective amount of the cells of claim 4.

14. The method of claim 13, wherein the cells are administered to a site on the subject experiencing hair loss in an amount effective to induce hair follicle formation.

15. The method of claim 14 wherein the hair loss is due to androgenetic alopecia, wounding, trauma, scarring, telogen effluvium, genetic pattern baldness or with hormonal disorders that decrease hair growth or cause loss of hair.

16. The method of claim 13 wherein the cells administered to the subject are autologous or allogeneic cells.

17. A method for detecting trichogenic dermal cells comprising
contacting a population of skin cells with a binding moiety specific for EGF latrophilin and seven transmembrane domain-containing protein 1 (ELTD1), Transmembrane Protein 108 (TMEM108), Hyaluronan and proteoglycan link protein 1 (HAPLN1); and
detecting the binding moiety on skin cells, wherein detection of the binding moiety on the skin cell is indicative of the skin cell being a trichogenic dermal cell.

18. The method of claim 17 wherein the binding moiety is an antibody or antigen-binding fragment thereof.

19. The method of claim 17 wherein the binding moiety is labeled with a detectable label.

20. The method of claim 19 wherein the detectable label is selected from the group consisting of a radioisotope, fluorophore, or enzyme.

21. The method of claim 17 wherein in the binding moiety is detected using immunological detection, spectrophotometry, fluorospectroscopy, or mass spectroscopy.

22. The method of claim 17 wherein the trichogenic dermal cells are isolated using a cell sorting method such as fluorescent-activated cell sorting (FACS) and/or magnetic bead cell sorting (MACS).

23. The method of claim 17 wherein the trichogenic dermal cells are isolated using a magnetic bead isolation.
